(19) United States
(12) Patent Application Publication
(10) Pub. No.: US 2008/0220512 A1
(43) Pub. Date: Sep. 11, 2008

(54) Tunable laser-based spectroscopy system for non-invasively measuring body water content

(75) Inventors: Seungug Koh, Sunnyvale, CA (US); Martin Debreczeny, Danville, CA (US); Clark R. Baker, Newman, CA (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC, Pleasanton, CA (US)

(21) Appl. No.: 11/716,394

(22) Filed: Mar. 9, 2007

(51) Int. Cl. C12M 3/00

(52) U.S. Cl. 435/288.7

(57) ABSTRACT

The present disclosure relates to a tunable laser-based spectroscopy system for accurately and non-invasively measuring body water content. The body water content is one of the important health indicators, by which one can quantitatively monitor the hydration level of body and determine if it is necessary to supplement or reduce the body water. The disclosed systems, devices, and/or methods may improve wavelength accuracy, wavelength resolution, optical spectral power density, signal-to-noise ration, and available implementation options for the spectroscopy system.

### TECHNICAL FIELD

The present disclosure is related to systems, devices, and methods for assessing and/or evaluating one or more body-fluid metrics.

### BACKGROUND

Dehydration may be associated with increased risk of developing dental disease, urinary tract infections, broncho-pulmonary disorders, kidney stones, constipation, poor immune function, cardiovascular pathologies, and impaired cognitive function. The maintenance of body fluid balance may often be one of the foremost concerns in the care and treatment of critically ill patients, yet physicians have access to few diagnostic tools to assist them in this vital task. Patients with congestive heart failure, for example, frequently suffer from chronic systemic edema, which must be controlled within tight limits to ensure adequate tissue perfusion and prevent dangerous electrolyte disturbances. Dehydration of infants and children suffering from diarrhea can be life-threatening if not recognized and treated promptly.

The most common method for judging the severity of edema or dehydration is based on the interpretation of subjective clinical signs (e.g., swelling of limbs, dry mucous membranes), with additional information provided by measurements of the frequency of urination, heart rate, urea nitrogen (BUN)/creatinine ratios, and blood

### SUMMARY

Therefore, there exists a need for methods and devices for monitoring body fluid (e.g., water) metrics that are less invasive, less subjective, and more accurate. The present disclosure, according to some specific example embodiments, relates to systems, devices, and/or methods for assessing body fluid-related metrics and changes therein. Other specific example embodiments, according to the present disclosure, further relate to systems, devices, and/or methods for correlating body fluid-related metrics, e.g., in a particular tissue with the corresponding whole-body metric.

The present disclosure relates to systems for assessing a body fluid metric. According to some embodiments, a system for assessing a body fluid metric may include a laser configured and arranged to illuminate at least a portion of a body tissue and a single detector in optical communication with the at least a portion of a body tissue. For example, a system may include a tunable laser and/or one or more fixed wavelength lasers. A system, in some embodiments, may include a sensor comprising a first optic fiber having a first end that is configured and arranged to optically communicate with the laser and having a second end that is configured and arranged to optically communicate with a tissue sample; and a second optic fiber having a first end that is configured and arranged to optically communicate with the detector and having a second end that is configured and arranged to optically communicate with a tissue sample. A system may further include a wavelength division multiplexer and/or an optical switch in optical communication with the laser and the first optic fiber. A system, according to some embodiments, may include a detector comprising a photodiode and/or an optical switch in optical communication with the second optic fiber. An optic fiber (e.g., a first optic fiber and/or a second optic fiber) may comprise a coupler in some embodiments. A sensor, according to some embodiments, may be configured and arranged to be disposable or reusable. A sensor may include a collimator, a star coupler, and/or a beam expander in optical communication with the laser. In some embodiments, a system may exclude a diffraction grating and/or a detector array.

In addition, the present disclosure relates to a sensor configured and arranged to releasably and operably contact a body fluid metric assessment system. A sensor, in some embodiments, may include a first optic fiber having a first end that is configured and arranged to optically communicate with the laser and having a second end that is configured and arranged to optically communicate with a tissue sample; and a second optic fiber having a first end that is configured and arranged to optically communicate with the detector and having a second end that is configured and arranged to optically communicate with a tissue sample. A sensor, according to some embodiments, may be configured and arranged to be disposable or reusable. In some embodiments, a sensor may include a wavelength division multiplexer, a collimator, and/or a beam expander in optical communication with the first optic fiber and configured and arranged to optically communicate with the laser. In some embodiments, a sensor may include an optical switch in optical communication with the second optic fiber and configured and arranged to optically communicate with the detector. In some embodiments, a sensor may include one or more optic fibers (e.g., a first optic fiber and/or a second optic fiber) having a coupler.

According to some embodiments, a sensor may be configured and arranged to releasably and operably contact a body fluid metric assessment system comprising a tunable laser and a processor and the sensor may include an optic fiber having a first end that is configured and arranged to optically communicate with the tunable laser and having a second end that is configured and arranged to optically communicate with a tissue sample; a photodiode configured and arranged to optically communicate with a tissue sample; and a wire having a first end in electrical communication with the photodiode and a second end configured and arranged to electrically communicate with the processor.

The present disclosure further relates to methods of assessing a body fluid metric. For example, a method of assessing a body fluid metric may include illuminating at least a portion of a body tissue with a laser; detecting at least one property (e.g., wavelength and/or absorption) of at least one wavelength of light emanating from the at least a portion of a body tissue using a single detector; and processing the at least one property of the at least one wavelength of light emanating from the at least a portion of a body tissue to produce a body fluid metric. A laser used in a method of assessing a body fluid metric may include a tunable laser and/or one or more fixed wavelength lasers. A method, in some embodiments, may include emitting light of a selected discrete wavelength from a tunable laser. A method, in some embodiments, may include emitting light from the laser toward an optical switch and/or a collimator and selecting a wavelength of light with which to illuminate the at least a portion of a body tissue using the optical switch.

A method, in some embodiments, may include emitting light from the laser toward a wavelength division multiplexer, splitting the emitted light into two or more beams using the wavelength division multiplexer, and illuminating a number of at least a portion of body tissues corresponding to the number of beams. A method, in some embodiments, may include emitting light from the laser toward an optical switch, splitting the emitted light into two or more beams using the optical switch, and illuminating a number of at least a portion of body tissues corresponding to the number of beams. According to some embodiments, the wavelength of light in each beam may be the same as or may differ from the wavelength of light in the other beams.

A method, in some embodiments, may include processing comprising comparing the detected at least one wavelength with a reference to form a comparison and using the comparison to determine the hydration status of the at least a portion of a body tissue. A reference may include, for example, at least a portion of an absorption spectra for a reference tissue. The comparing to form a comparison, in some embodiments, may further include evaluating the difference between the detected at least one wavelength and the reference. The evaluating may further include quantitatively and/or qualitatively evaluating the difference between the detected at least one wavelength and the reference in some embodiments. An absorption spectrum for a reference tissue (e.g., a normally hydrated tissue) may be from about 1450 nm to about 1650 nm. This may correspond to a prominent water absorption band. In some embodiments, an increase in the absorption at the detected at least one wavelength in this band relative to the reference may indicate over-hydration. In some embodiments, an increase in the wavelength(s) at which the tissue absorption is equal to the absorption of the reference at one or more predetermined wavelengths within this band may indicate over-hydration. In some embodiments, a decrease in the absorption at the detected at least one wavelength in this band relative to the reference may indicate dehydration. In some embodiments, a decrease in the wavelength(s) at which the tissue absorption is equal to the absorption of the reference at one or more predetermined wavelengths within this band may indicate dehydration.

### BRIEF DESCRIPTION OF THE DRAWINGS

Some specific example embodiments of the disclosure may be understood by referring, in part, to the following description and the accompanying drawings, wherein:

FIG. 1 is a bar graph of water content as a percentage of total mass and lean mass for men and women between the ages of 20 and 79 (adapted from S H Cohn et al., J Lab. Clin. Med. 105(3): 305-311 (1985));

FIG. 2 is a bar graph of water content as a percentage of fat-free mass and fat-free-bone-free mass for men and women between the ages of 20 and 79 (adapted from S H Cohn et al., J Lab. Clin. Med. 105(3): 305-311 (1985));

FIG. 3 is a graph of the correlation between separate fat-free or lean water fraction (“f_{w}^{L}”) measurements on the same subject;

FIG. 4A shows an isometric view of one example of a system with a disposable water probe in an engaged position according to the teachings of the present disclosure;

FIG. 4B shows a cut-away view of the water assessment system of FIG. 4A with the disposable water probe in a disengaged position;

FIG. 5A shows an isometric view of one example of a system with a disposable water probe in an engaged position according to the teachings of the present disclosure;

FIG. 5B shows a cut-away view of the system of FIG. 5A with the disposable water probe in a disengaged position;

FIG. 5C shows an isometric view of a variation of a system of FIG. 5A in which the monitor is separate from the base unit and the disposable water probe is in contact with the mid-line of the torso of a subject;

FIG. 6 shows the results of measuring lean water fraction (f_{w}^{l})in tissue biopsies taken at different elevations;

FIG. 7 shows the optical f_{w}^{l} estimates with data gathered simultaneously with the tissue biopsies;

FIG. 8A shows absorption spectra of a normal subject and an over-hydrated subject (red-shifted);

FIG. 8B shows absorption spectra of a normal subject and a dehydrated subject (blue-shifted);

FIG. 8C shows absorption spectra of a normal, hydrated subject;

FIG. 9A shows an example sensor configuration according to an embodiment of the disclosure;

FIG. 9B shows an example sensor configuration in which a tunable laser and a detector are within a common housing and fibers convey light to and from a tissue according to an embodiment of the disclosure;

FIG. 9C shows an example sensor configuration in which a tunable laser and a detector are within a common housing and a fiber convey light to a tissue and diffusely reflected light is collected by a photodiode and conveyed as an electric signal to a processor according to an embodiment of the disclosure;

FIG. 10A shows an example tunable laser configuration in which light from a series of tunable lasers (designated 1 to N) is conveyed to a common wavelength division multiplexer (WDM) according to an embodiment of the disclosure;

FIG. 10B shows an example tunable laser configuration in which light from a series of tunable lasers (designated 1 to N) is conveyed to a common optical switch according to an embodiment of the disclosure;

FIG. 11A shows an example sensor structure comprising a collimator and/or a beam expander and an array of fibers according to an embodiment of the disclosure;

FIG. 11B shows an example sensor structure comprising a star coupler and a collimator and/or a beam expander according to an embodiment of the disclosure;

FIG. 12A shows an example multi-site sensor structure comprising a single tunable laser, a star coupler and a series of detectors (numbered 1 to N) according to an embodiment of the disclosure;

FIG. 12B shows an example multi-site sensor structure comprising a single tunable laser, an optical switch, and a series of detectors (numbered 1 to N) according to an embodiment of the disclosure; and

FIG. 12C shows an example multi-site sensor structure comprising a single tunable laser, a pre-tissue optical switch, a post-tissue optical switch, and a single detector according to an embodiment of the disclosure.

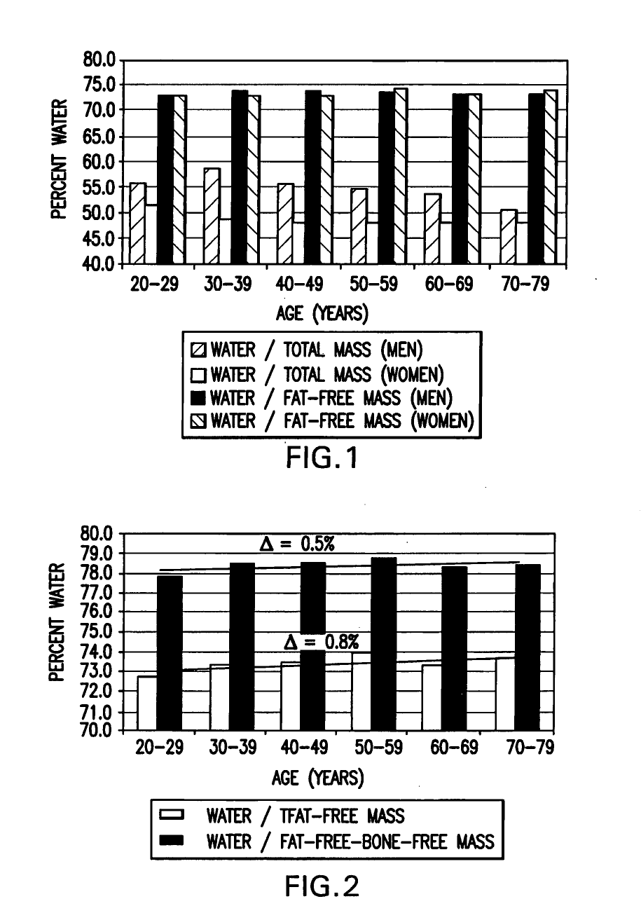

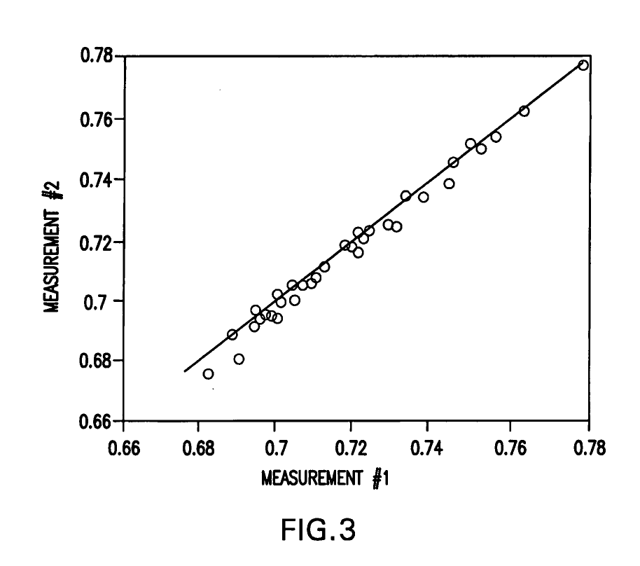

While the invention is susceptible to various modifications and alternative forms, specific embodiments have been shown by way of example in the drawings and will be described in detail herein. However, it should be understood that the invention is not intended to be limited to the particular forms enclosed. Rather, the invention is to cover all modifications, equivalents and alternatives falling within the spirit and scope of the invention as defined by the appended claims.

### DETAILED DESCRIPTION

The present disclosure relates to systems, devices, and methods for assessing a water metric in a subject, tissue, or portion thereof.

According to some specific example embodiments, systems, devices, and/or methods of the disclosure may be useful in assessing, monitoring, and/or adjusting fluid, e.g., water, status in a subject or in any portion of a subject. For example, various fluids, as well as diuretics, are commonly administered multiple times to patients in surgery or intensive care without assessing patient hydration. In the absence of such feedback, the patient may be exposed to a risk of inadequate or excessive local and/or systemic hydration. According to another specific example embodiment, systems, devices, and/or methods of the disclosure may contribute to minimizing this risk through assessment of one or more body-fluid related metrics. According to a further specific example embodiment, the systems, devices, and/or methods of the disclosure may be applied to any tissue and/or region of the subject's body. Body-fluid metrics may be assessed in any multicellular organism and/or any portion of a multicellular organism. In some specific example embodiments, a subject may be a mammal (or other animal). In some specific example embodiments, a mammalian subject may be human.

Water may generally partition into one of two compartments in the body, namely, inside cells (the intracellular compartment) and outside of the cells (the extracellular compartment). The extracellular compartment is further divided into vascular and interstitial compartments. According to a specific example embodiment, systems, devices, and/or methods of the disclosure may allow assessment of body-fluid related metrics including, without limitation, (a) total water fraction (f_{w}^{T}), (b) fat-free and/or lean tissue water fraction (f_{w}^{L}), (c) intravascular water fraction (f_{w}^{IV}), (d) extravascular water fraction (f_{w}^{EV}), (e) interstitial water fraction (f_{w}^{IS}), (f) intracellular water fraction (f_{w}^{IC}), (g) extracellular water fraction (f_{w}^{EC}), and/or (h) combinations thereof.

Several methods of quantitating fluid, e.g., water, in the body may be used in accordance with the teachings of the present disclosure including, without limitation, bioimpedance, transepidermal water loss, viscoelastic measurements, dielectric conductance, optical spectrophotometry, magnetic resonance, ultrasound, and/or combinations thereof. For example, bioimpedance analysis and/or bioelectrical impedance spectroscopy may be used to apply an electrical current to assess tissue conductivity and, thereby, obtain a local and/or systemic fluid metric.

Any tissue site of the subject's body may be assessed using the systems, devices, and/or methods of the disclosure. In some specific example embodiments, the fluid, e.g., water, content of a site at or near the surface of the skin may be assessed. In other specific example embodiments, the water content of localized tissue sites at the surface of or within an organ may be assessed. Organs that may be assessed include, without limitation, the brain, the eyes, the nose, the mouth, the esophagus, the stomach, the skin, the intestines, the liver, the gall bladder, the pancreas, the spleen, the heart, blood, the lungs, the kidneys, the liver, the vagina, the cervix, the uterus, the fallopian tubes, the ovaries, the penis, the testes, the prostate, the bladder, and/or the pancreas. Tissues that may be assessed include, without limitation, muscles, bones, adipose, tendons, and/or ligaments.

Liquid water has an absorption spectrum that includes a spectral band covering from 1350 nm to 1650 nm as well as absorption spectral bands in both shorter and longer wavelength ranges. According to some embodiments of the disclosure, one or more wavelengths in spectral bands outside the 1350-1650 nm band may be used to estimate hydration.

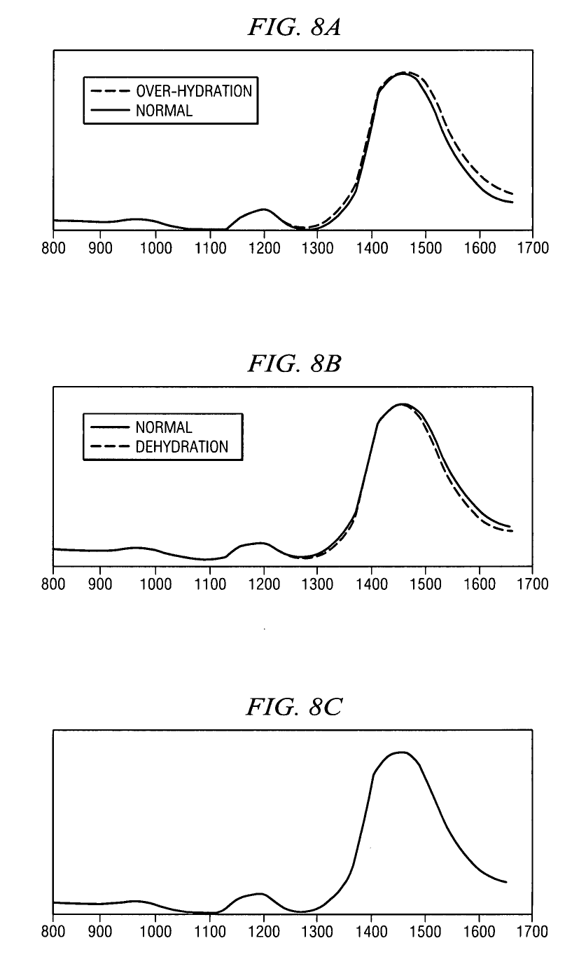

Measured spectra in piglets has revealed a broadening or narrowing absorption spectra between 1350 nm and 1650 nm in response to hydration levels. Absorption spectra in the above wavelength range are attributed to light absorption by water. It has been observed in animal studies that long wavelengths of water absorption bands may change depending on an animal's water status. Specifically, it has been observed that long wavelengths of water absorption bands were shifted toward the right (red-shift) in over-hydrated piglets (FIG. 8A) and was shifted toward the left (blue-shift) in dehydrated piglets (FIG. 8B). These red-shifts and blue-shifts have resulted in broadening or narrowing the measured spectra. The shift direction of the right tail has been consistently related to the hydration levels in both dehydration and over-hydration measurements.

In some embodiments, the magnitude of the right tail (i.e., long wavelength) shift (red-shift or blue-sift) may be about 20 nm relative to a right tail of reference spectra. A reference spectra may be a spectra obtained from a specimen that is neither dehydrated nor over-hydrated (e.g., FIG. 8C). A right tail, in some embodiments, may be or may include the absorption spectra between about 1450 nm and about 1650 nm. The spectral shift in the bottom trunk portion of spectral envelope may be larger than the shift in the peak portion of the spectral envelope.

A shift in absorption may be assessed in one of at least two ways. For example, the absorption of light of a given wavelength by a test tissue may be compared with the absorption of light at that same wavelength by a reference tissue. An increase in absorption by the test tissue over the reference (e.g., where the reference is normally hydrated tissue) may be correlated with a condition of over-hydration in the test tissue. Similarly, a decrease in absorption by the test tissue over the reference (e.g., where the reference is normally hydrated tissue) may be correlated with a condition of dehydration in the test tissue.

According to another example for assessing a shift in absorption, the absorption of light of a given wavelength (e.g., a detected wavelength) by a test tissue may be compared with a reference spectrum, a wavelength in the reference spectrum having the same absorption may be identified, and the difference between the given wavelength and the identified reference wavelength may be assessed. A condition of over-hydration in the test tissue may exist where the given wavelength is higher than the identified reference wavelength (e.g., where the reference is normally hydrated tissue). A condition of dehydration in the test tissue may exist where the given wavelength is lower than the identified reference wavelength (e.g., where the reference is normally hydrated tissue).

Without being limited to any specific mechanism of action, weak hydrogen bonds available in water molecules might play a role in broadening or narrowing the observed absorption spectra ranging from 1350 nm to 1650 nm. In gaseous and sparsely populated states, one may observe narrow and well-defined absorption spectra of water vapors. But, in liquid states, water molecules interact closely with each other through relatively weak hydrogen bonds, which may cause the covalent O—H bonds within each water molecule to occupy a very broad level of energy states. In general, the larger or broader the density of states for the molecular bonding energies, the wider the molecular absorption spectra becomes. The presence of other species in the liquid water may play an important role in influencing the level of weak hydrogen bonds among the water molecules too, which, in turn, may alter the absorption spectra of such mixtures. Some published journal articles, e.g., “Estimation of concentration and bonding environment of water dissolved in common solvents using near infrared absorptivity” by B. Dickens and S. H. Dickens, Journal of Research of the National Institute of Standards and Technology, Vol. 104, No. 2, March-April 1999, have reported that the presence of other species in the liquid water has narrowed the water absorption spectra. The presence or absence of these tight bonds with other species in liquid water may reduce or increase the available hydrogen bonds in the liquid water and may result in either red-shifts or blue-shifts of the absorption spectra.

Without being limited to any specific mechanism of action, the following hypotheses may explain observed phenomena. In some embodiments, if only water is lost from a tissue during dehydration, the effect may be regarded as a decrease in the concentration of water and/or an increase in the concentration of the remaining chemical species in the tissue. The increased concentration of these remaining species may correlate with an increase in the number of water-to-other-species bonds and/or a decrease in the number of water-to-water hydrogen bonds. A decreased level of hydrogen bonds in the liquid water may reduce the range of energy states for the covalent bonds in the liquid water and the absorption spectra will be narrowed (blue-shifts) accordingly. On the other hand, if water alone is added during hydration (e.g., over-hydration), the effect may be regarded as an increase in the concentration of water and/or a decrease in the concentration of the remaining chemical species in the tissue. A decreased concentration of other species may correlate with a decrease in the number of water-other species bonds and/or an increase in the number of water-water hydrogen bonds. An increased level of hydrogen bonds in the liquid water may increase the range of energy states for the covalent bonds in the liquid water and the absorption spectra will be broadened (red-shifts) accordingly.

Regardless of the correctness of the above hypotheses, if the absorption spectra broadens or narrows according the hydration level of optically probed tissues, then it may be possible to estimate the subjects' hydration level, e.g., by calculating the amount of spectral shifts. A sensitive and accurate spectroscopy measurement may be desired or required in measuring the spectra response of test subject. Hence, improved wavelength resolution, wavelength stability, optical spectral power density, and signal-to-noise ratio of the detected lights may be desired.

### Systems and Devices

In some specific example embodiments, the present disclosure provides systems and devices for measuring a body-tissue fluid content metric, e.g., water content. Systems and/or devices for assessing whole-body water content of a subject may include a local water content probe (e.g., a water probe) configured to assess a local fluid metric at a tissue site (e.g., a tissue site of interest and/or a tissue reference site). A system, device, and/or probe of the disclosure may include, in some embodiments, a reflectance standard (e.g., a Teflon block) to calibrate out the wavelength response or sensitivity or emissivity of tissue site emitters and/or detectors. A system and/or device of the disclosure may include, in some embodiments, a probe receiver configured to contact an area at or near a tissue site and configured to releasably engage a probe and/or probe housing. For example, a probe receiver may include a toroidally-shaped adhesive pad that encircles a tissue site when positioned on a subject and receives a probe or probe housing into its center space.

Systems and/or devices of the disclosure may also include a tissue compressor configured to alter (e.g., increase or decrease) the hydrostatic pressure of an assessment site and/or a reference site. Systems and/or devices of the disclosure may further include a probe location information sensor configured to determine location information of a probe and/or an assessment site. Systems and/or devices according to the disclosure may further include a processor, e.g., a processing device, configured to process a local fluid content metric at a tissue site of interest and a local fluid content metric at a tissue reference site to produce a whole-body fluid content metric. In other specific example embodiments, systems and/or devices according to the disclosure may further include a processor, e.g., a processing device, configured to process a local fluid content metric and probe location information to produce a whole-body fluid content metric. According to some specific example embodiments, at least a portion of a system or device of the disclosure may be configured to be sterile, sanitizable, disposable, replaceable, and/or repairable. In other specific example embodiments, at least a portion of a system and/or device, e.g., a probe, may be covered with a disposable cover. For example, a probe may be covered in whole or in part by a hygienic cover similar to those used with infrared ear thermometers.

Systems and/or devices of the disclosure may be configured to assess a local fluid metric by any means available including, without limitation, bioimpedance, transepidermal water loss, viscoelastic measurements, optical spectrophotometry, magnetic resonance, ultrasound, and/or combinations thereof. For example, systems and/or devices may be designed to make measurements using optical spectrophotometry. A device may include a probe housing configured to be placed near and/or at an assessment site; light emission optics connected to the housing and configured to direct radiation at the assessment site; and/or light detection optics connected to the housing and configured to receive radiation from the assessment site. A system may include a probe housing configured to be placed near and/or at an assessment site; light emission optics connected to the housing and configured to direct radiation at the assessment site; light detection optics connected to the housing and configured to receive radiation from the assessment site; a processing device, e.g., a processor, configured to process radiation from the light emission optics and the light detection optics to compute the metric; and/or a display on which raw data and/or the body-fluid metric may be displayed. The display may be operably coupled to light emission optics, light detection optics, and/or a processor. A device, e.g., a probe housing, may include a pressure transducer to assess the compressibility of tissue for deriving an index of a fraction of free water within said tissue.

According to some specific example embodiments, systems and/or devices may include a light source capable of emitting electromagnetic radiation of at least one wavelength. For example, systems and/or devices may include a light source that emits a broad or narrow band of wavelengths of infrared, visible, and/or ultraviolet light. The light source may also emit fluorescent or phosphorescent light. A light source may emit light continuously, intermittently and/or sporadically. In some specific example embodiments, systems and/or devices may include any additional spectrophotometry components including, without limitation, one or more modulators, polarizers, rhombs, etalons, prisms, windows, gratings, slits, interferometers, lenses, mirrors, reflective phase retarders, wavelength selectors, waveguides, beam expanders, beam splitters, and/or photodetectors.

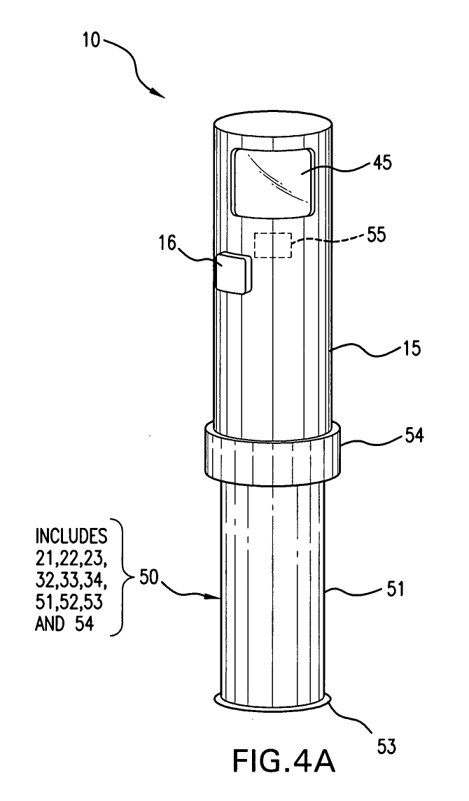

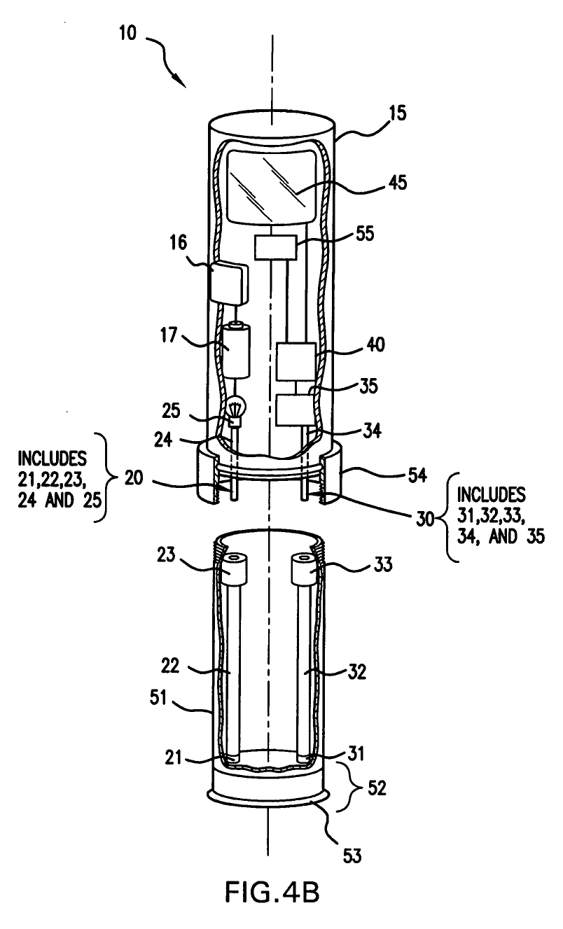

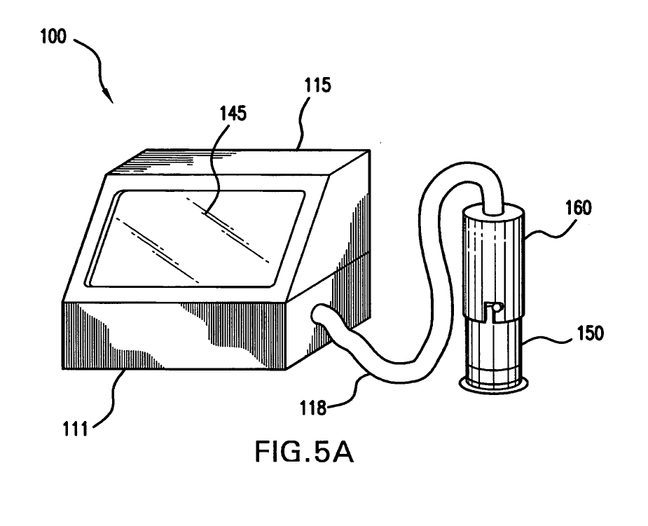

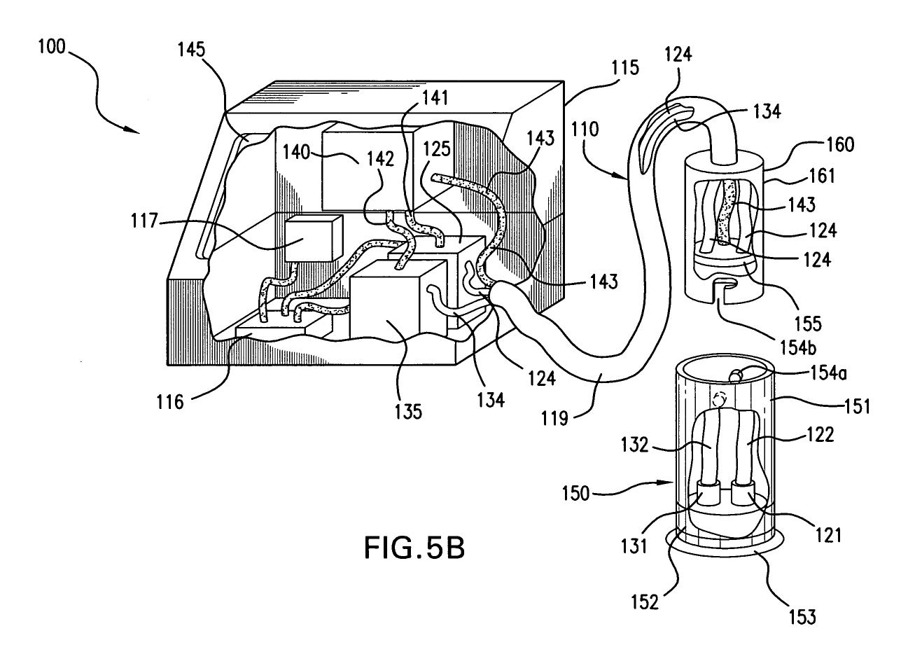

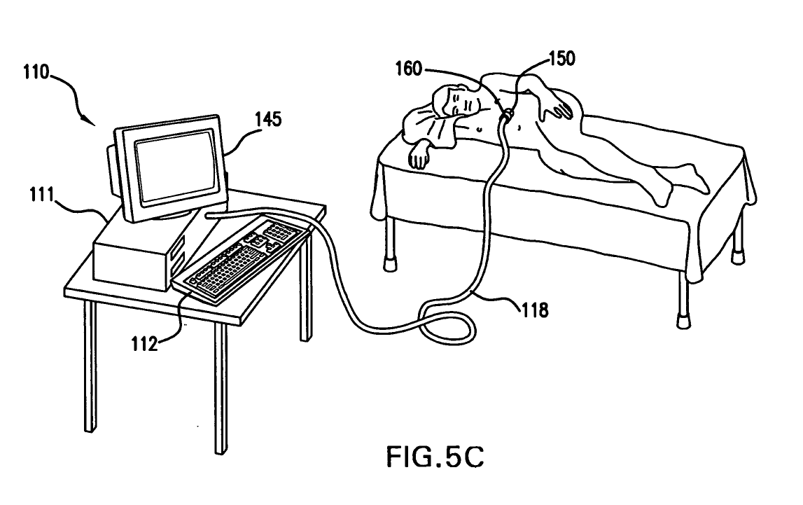

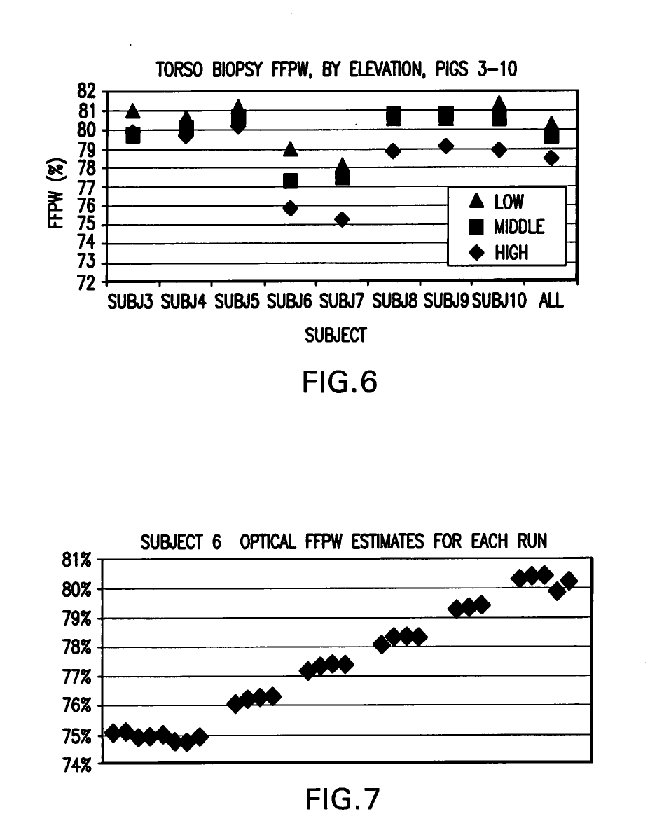

Some specific example embodiments of the disclosure may be understood by reference, in part, to FIGS. 4A-5C, wherein like numbers refer to same and like parts. These figures are illustrative only and are not intended to limit the possible sizes, shapes, proportions, and/or relative arrangements of various specific example embodiments. Table 1 lists reference numerals with their associated names and figures in which they appear.

TABLE 1

FIG.

| | 4A, 4B | 5A, 5B, 5C |
|---|---|---|
| system | 10 | 110 |
| base unit | | 111 |
| keyboard | | 112 |
| housing | 15 | 115 |
| trigger | 16 | |
| controller | | 116 |
| battery | 17 | |
| power inlet or source | | 117 |
| optical fiber bundle | | 118 |
| optical fiber bundle housing | | 119 |
| light emission optics | 20 | 120 |
| light emission aperture | 21 | 121 |
| disposable fiber optic cable | 22 | 122 |
| fiber optic cable connector | 23 | 123 |
| fiber optic cable | 24 | 124 |
| light source | 25 | 125 |
| light detection optics | 30 | 130 |
| light detection aperture | 31 | 131 |
| disposable fiber optic cable | 32 | 132 |
| fiber optic cable connector | 33 | 133 |
| fiber optic cable | 34 | 134 |
| light detector | 35 | 135 |
| processor | 40 | 140 |
| processor connector | | 141 |
| processor connector | | 142 |
| processor connector | | 143 |
| display | 45 | 145 |
| water probe | 50 | 150 |
| water probe housing | 51 | 151 |
| spacer | 52 | 152 |
| seal | 53 | 153 |
| water probe connector | 54 | 154 |
| connector tab | | 154a |
| connector groove | | 154b |
| water probe location sensor | 55 | 155 |
| location sensor connector | | 156 |
| probe manipulator | | 160 |
| probe manipulator housing | | 161 |

In some example embodiments, a spectroscopy system may include transmission, reflectance, fluorescence, absorption, and Raman spectroscopy. Light emitted toward a test sample may be filtered (e.g., diffracted) to allow analysis of one or more distinct wavelengths. Light filtration may occur before (pre-filter) or after (post-filter) emitted light reaches a test sample. According to some specific example embodiments, a broadband white light source may be used. A broadband white light source, in some embodiments, may produce a low optical spectral power density.

In some specific example embodiments, pre-filtration may be constrained by an inability to simultaneously generate a plurality of filtered outputs and/or an inability to produce a continuously variable filtered output. Poor signal-to-noise ratio may limit the performance of post-filtration systems. In addition, broadband light source efficiency may be quite low since the light outside of the spectra of interest may be wasted in the measurement. Therefore, a spectroscopy system with a higher optical spectral power density, a higher source efficiency, a higher signal-to-noise ratio, a capacity to generate a plurality of filtered outputs, and/or a capacity to generate a continuously variable output may be desired and/or needed.

In some embodiments, a spectroscopy system may include a fixed-wavelength laser and/or a tunable laser (e.g., as a light source). A tunable laser may be configured and arranged to interface with any optical device (e.g., a fiber optic device). For example, a tunable laser may be configured and arranged to be in optical communication with an optical fiber, a wavelength division multiplex (WDM) filter, an optical switch, an optical modulator, a variable attenuator, an optical circulator, a tunable filter, a fiber collimator, a coupler, and combinations thereof. These fiber optic devices may improve the functionality and/or performance of a prior art spectroscopy system.

According to some embodiments, a laser may generate an optically coherent beam. An optically coherent beam may be temporally and/or spatially coherent. A laser may produce highly focused and highly monochromatic light. Selective amplification of stimulated emission inside the laser cavity may produce a beam with an extremely narrow linewidth (i.e., quasi-monochromatic light). So, unlike incoherent light sources, a laser may generate light output primarily or exclusively at a desired wavelength of interest or wavelength band of interest (e.g., there may be little or no wasted light output).

Due to the narrow linewidth, the optical spectral power density of laser may be many orders higher than that of incoherent light sources. A laser-based light source may also provide good wavelength accuracy and a high wavelength resolution compared with other light sources. A laser may be modulated at very high speed either directly or externally. A laser may be sensitive to an operation temperature and its lifetime may be shorter than the lifetime of an LED.

Tunable lasers represent a special class of lasers which may continuously tune its output wavelengths by changing an optical cavity boundary condition. For example, the tunable lasers for a dense-wavelength-division-multiplexer (DWDM) optical network routinely tune the emission central wavelength of the emitted lights in excess of 40 nm range with a 25 GHz, 50 GHz, or 100 GHz channel spacing accuracy. A tunable laser may have one or more of the following: a few MHz linewidth, +9 dBm output power, 200 nm wavelength range, 80 nm/s sweep speed, +60 dB side-mode suppression ratio, ±0.01 dB power stability, and/or better than 10 pm wavelength accuracy. In telecommunication applications, tunable laser sources are available in the wavelength bands covering from 1260 nm to 1640 nm. And the lasers can generally be tuned to a different emission wavelength as long as the laser cavity boundary condition can be modified accordingly without causing any adverse physical effects.

In some specific example embodiments, a tunable laser-based spectroscopy system may exclude a diffraction grating and/or may exclude a detector array since the wavelength band is selected by the continuously tunable laser light source. For example, a single detector may be used in place of an entire array of detectors. Alternatively, a grating or detector array may be included in some embodiments. For example, a detector array may be used to probe multiple sites/samples simultaneously (e.g., FIG. 12B). A grating may be used, for example, to combine multiple lasers (e.g., FIG. 10A). Also, a grating may be used, for example, to increase the spectral resolution of a laser or other light source (e.g., a light emitting diode).

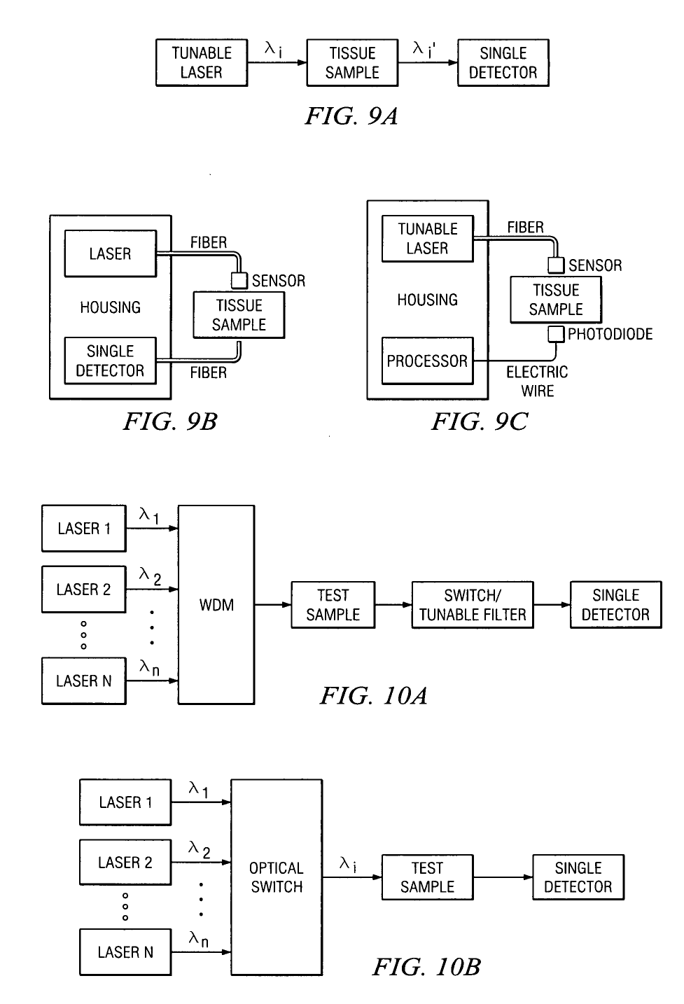

The high optical spectral power density out of the tunable laser may permit implementation of a spectroscopy system in a wide variety of configurations. In some embodiments, a spectroscopy sensor may be configured by using optical fibers only, where the delivery of light signals into and from the test subjects may be handled entirely by input and output optical fibers (FIG. 9B). This configuration may permit placing both light source and light detector inside a common housing (e.g., a monitor unit) away from the sensor unit. In some embodiments, a system and/or device may include a fiber-cable hybrid configuration (FIG. 9C). In the fiber-cable hybrid configuration, light signals from the test subjects may be coupled directly into the detector, which may generate a corresponding electrical signal to be transferred by the cable wires. A detector may be photodiode, photo-transistor, photo-conductor, photo-cell, and any other optical-to-electrical transducer capable of converting the signal form from an optical to an electrical domain. Note that the high optical spectral power density of a tunable laser may improve a signal-to-noise ratio of detected signal(s) and may permit many useful embodiments of the current disclosure.

A detector, according to some embodiments, may be placed in a temperature controlled mount (e.g., a thermo-electric (TE) cooler). In some embodiments, a detector may exclude a temperature controlled mount and operate instead as a room temperature device. So the improved signal-to-noise ratio offered by some embodiments of the present disclosure may allow the use of room temperature detector and result in a simple and low-cost detector.

In another embodiment, sensors may be distributed to multiple sites from a single light source. A single light source may be distributed to multiple test subjects or sites passively by using one-input-to-multiple-output optical coupling devices like a star coupler (e.g., FIG. 12A). Alternatively a single light source may be actively switched among the test subjects or sites by using an optical switch too (e.g., FIG. 12B).

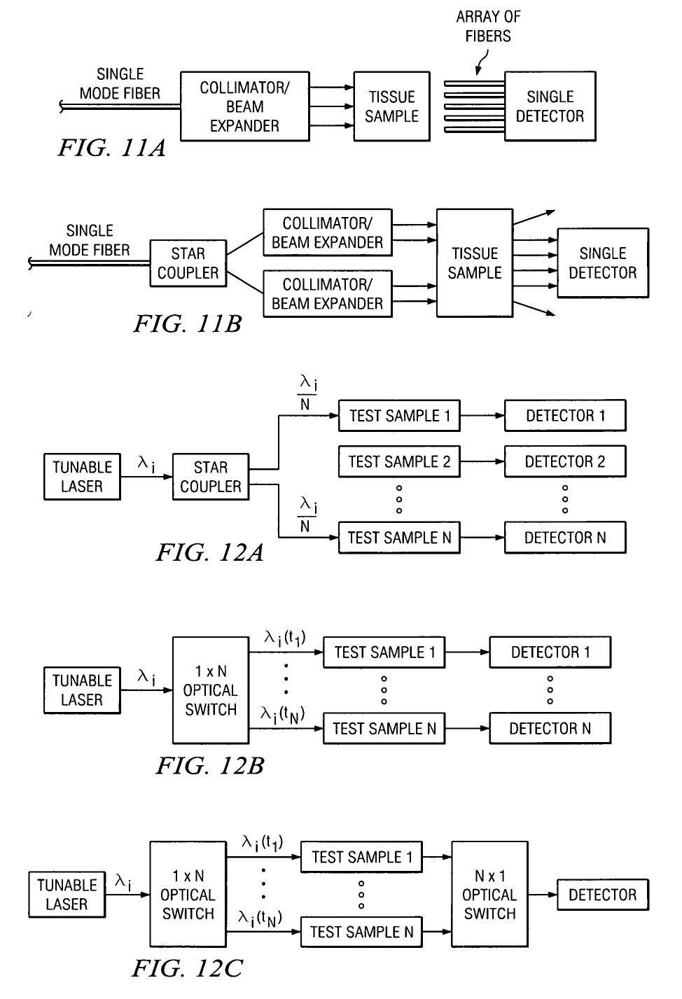

A tissue sample may be illuminated with one or more light outputs according to some embodiments. For example, a tissue sample may be illuminated simultaneously by the output of a plurality of fixed wavelength lasers that have been combined into a single fiber port using a WDM filter (e.g., FIG. 10A). Alternatively, a tissue sample may be illuminated sequentially by the output of a plurality of fixed wavelength lasers using an optical switch (e.g., FIG. 10B). And, if there is a need to change the laser wavelengths, tunable lasers may be used instead of fixed-wavelength lasers. In another embodiment, an optical beam shaping device (e.g., a collimator, beam expander, diffuser, and/or pattern generator) may be used to illuminate a test subject (e.g., FIGS. 11A and 11B). It is also possible to insert a variable optical attenuator along the optical path in order to control the intensity of illuminated optical beam. In the distributed sensor array configuration, the multiplicity of detectors can be replaced by a single detector if an optical switch is employed.

According to some embodiments of the disclosure a system may include one or more tunable light sources, and one or more sensors. A tunable light source may emit one or more wavelengths of light in the direction of a sample. A system, in some embodiments, may be configured and arranged to assess one or more properties of light emitted toward or received from a sample. For example, a tunable light source may also be configured to assess one or more properties of the light emitted toward the sample. A sensor may be configured and arranged to receive one or more wavelengths of light emanating from a sample. In addition, a sensor may be configured and arranged to assess one or more properties of light received from a sample. A sensor, in some embodiments, may include a fiber input and/or a fiber output. For example, a fiber input may be optically coupled to a tunable light source and/or a sample. A fiber input may also be configured to assess one or more properties of light delivered to a sample. Similarly, a fiber output may be optically coupled to a sample and/or a detector (e.g. a photo diode). A fiber output may also be configured to assess one or more properties of light emanating from a sample. A sensor in some embodiments of the disclosure may include a fiber and/or a cable. For example, a sensor may include a fiber input and an electrical cable output. In some embodiments of the disclosure a fiber input may include one or more single-mode fibers and/or one or more multi-mode fibers. A fiber input may further include a collimator, a beam expander and/or a coupler. According to some embodiments of the disclosure a tunable laser may emit a single wavelength to a sample and a sensor may detect a single wavelength (or a plurality of wavelengths) emitted by the sample. For example, a tunable laser may emit a wavelength λᵢ and a detector may detect a wavelength Λ_{Iprime}. The wavelength emitted may be selected accurately and a tunable laser may be configured to emit wavelengths at high resolution. A tunable laser may be configured and arranged to emit a plurality of wavelengths of light simultaneously or in series.

### EXAMPLE 1

Dehydration and over-hydration experiments were performed with a number of piglet subjects with similar starting weights. Dehydration was performed by removing water using a blood dialysis machine. Specifically, 350 mL of water was removed in the first 20 minutes of each hour. Typically, a total of 1750 mL water was removed from the piglet subjects by the end of dehydration experiments.

Similarly the piglets in the over-hydration group were subjected to an over-hydration process by supplementing a predetermined portion of water into the circulatory systems of piglets. Animals each received one liter of Ringer's lactate solution over 20 minutes of each hour for five hours.

Throughout the experiment, fat-free-percent-water (FFPW), an indicator of hydration level, was continuously estimated by optical spectroscopy according to an embodiment of the disclosure. At the end of each hour, tissue samples of piglets were obtained at various but predetermined piglet abdominal sites. Other relevant physiological parameters, such as temperature, pulse rate, oxygen saturation percentage, respiration rate, blood pressure, and body weight were monitored throughout the experiment.

The FFPW values may be estimated by identifying the linear combination of pure component spectra that best describe a measured tissue spectrum. For example, spectra for water, lipid, protein, hemoglobin, and oxyhemoglobin may be measured and utilized as basic component reference spectra. For the piglet experiments described in this example, the spectrometer's full-width-half-maximum resolution bandwidth was measured to be about 18 nm and the wavelength accuracy was specified to be about 0.2 nm by using Argon lamp reference spectra. The background dark current noise was removed from each spectrum measurement by subtracting it from the measured data. The intensity reference was measured by using a gold mirror or Teflon™ (polytetrafluoroethylene) blocks and the measured references were used as the intensity calibration reference in calculating the absorption spectra intensity.

The FFPW values may be estimated by using the entire spectra or by using a set of discrete wavelength points. For example, FFPW values were estimated by using a first set of wavelengths comprising 860 nm, 910 nm, 1110 nm, 1420 nm, and 1520 nm or a second set of wavelengths comprising 870 nm, 930 nm, 1420 nm, 1520 nm, and 1600 nm. Throughout both dehydration and over-hydration experiments, the FFPW estimation values from the measured spectra were consistent with the administered hydration levels for the piglet subjects. According to some embodiments, a 2-wavelength set may include 1390 nm and 1680 nm, a 3-wavelength set may include 1380 nm, 1680 nm, and 1835 nm and a 4-wavelength set may include 1395 nm, 1640 nm, 1665 nm, and 1835 nm. According to some embodiments, a 2-wavelength set may include 1392 nm and 1680 nm, a 3-wavelength set may include 1383 nm, 1682 nm, and 1838 nm and a 4-wavelength set may include 1397 nm, 1642 nm, 1667 nm, and 1687 nm.

As will be understood by those skilled in the art with the benefit of the instant disclosure, other equivalent or alternative methods for the measurement of the water fraction within tissue (f_{w}), as well as shifts in fluid between the intravascular and extravascular compartments, IVF-EVF or Q, according to embodiments of the present disclosure can be envisioned without departing from the essential characteristics thereof. For example, devices of the disclosure may be manufactured in either a handheld or a tabletop configuration, and may be operated sporadically, intermittently, and/or continuously. Moreover, individuals skilled in the art of near-infrared spectroscopy with the benefit of the instant disclosure would recognize that additional terms may be added to the algorithms used herein to incorporate reflectance measurements made at additional wavelengths and thus improve accuracy further. Also, light sources or light emission optics other than lasers including and not limited to incandescent light and narrowband light sources appropriately tuned to the desired wavelengths and associated light detection optics may be placed within the probe housing which may be placed near the tissue location or may be positioned within a remote unit; and which deliver light to and receive light from the probe location via optical fibers. Additionally, optical detectors may function in a forward-scattering mode, a back-scattering mode, a reflection mode, and/or a transmission mode. At least a portion of a system or device of the disclosure may be configured and arranged to be disposable, repairable, restorable, and/or sterilizible. The portion so configured may be a sensor or it may be a sensor cover. A laser of the disclosure may be tunable or may have a fixed output. Systems and devices of the disclosure may be configured and arranged to be portable (e.g., handheld units for field use) or relatively immobile (e.g., desktop or bench-top units for clinical use). Moreover, one of ordinary skill in the art with the benefit of the instant disclosure will appreciate that no embodiment, use, and/or advantage is intended to universally control or exclude other embodiments, uses, and/or advantages. These equivalents and alternatives along with obvious changes and modifications are intended to be included within the scope of the present disclosure. Accordingly, the foregoing disclosure is intended to be illustrative, but not limiting, of the scope of the disclosure as illustrated by the following claims.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. A system for assessing a body fluid metric, said device comprising:
a laser configured and arranged to illuminate at least a portion of a body tissue and a single detector in optical communication with the at least a portion of a body tissue.

2. A system according to claim 1 wherein the laser is a tunable laser.

3. A system according to claim 1 wherein the laser comprises one or more fixed wavelength lasers.

4. A system according to claim 1 further comprising a sensor comprising:
a first optic fiber having a first end that is configured and arranged to optically communicate with the laser and having a second end that is configured and arranged to optically communicate with a tissue sample; and
a second optic fiber having a first end that is configured and arranged to optically communicate with the detector and having a second end that is configured and arranged to optically communicate with a tissue sample.

5. A system according to claim 4 further comprising a wavelength division multiplexer in optical communication with the laser and the first optic fiber.

6. A system according to claim 4 further comprising an optical switch in optical communication with the laser and the first optic fiber.

7. A system according to claim 4 wherein the detector comprises a photodiode in optical communication with the second optic fiber.

8. A system according to claim 4 wherein the detector comprises an optical switch in optical communication with the second optic fiber.

9. A system according to claim 4 wherein the first optic fiber comprises a coupler.

10. A system according to claim 4 wherein the second optic fiber comprises a coupler.

11. A system according to claim 1 wherein the sensor is disposable.

12. A system according to claim 1 further comprising a collimator in optical communication with the laser.

13. A system according to claim 1 further comprising a beam expander in optical communication with the laser.

14. A system according to claim 1 further comprising a star coupler in optical communication with the laser.

15. A system according to claim 1 wherein the system exclude a diffraction grating.

16. A system according to claim 1 wherein the system excludes a detector array.

17. A sensor configured and arranged to releasably and operably contact a body fluid metric assessment system comprising a laser and a detector, said sensor comprising:
a first optic fiber having a first end that is configured and arranged to optically communicate with the laser and having a second end that is configured and arranged to optically communicate with a tissue sample; and
a second optic fiber having a first end that is configured and arranged to optically communicate with the detector and having a second end that is configured and arranged to optically communicate with a tissue sample.

18. A sensor according to claim 17 wherein the sensor is disposable.

19. A sensor according to claim 17 further comprising a wavelength division multiplexer in optical communication with the first optic fiber and configured and arranged to optically communicate with the laser.

20. A sensor according to claim 17 further comprising an optical switch in optical communication with the first optic fiber and configured and arranged to optically communicate with the laser.

21. A sensor according to claim 17 further comprising an optical switch in optical communication with the second optic fiber and configured and arranged to optically communicate with the detector.

22. A sensor according to claim 17 further comprising a collimator in optical communication with the first optic fiber and configured and arranged to optically communicate with the laser.

23. A sensor according to claim 17 further comprising a beam expander in optical communication with the first optic fiber and configured and arranged to optically communicate with the laser.

24. A sensor according to claim 17 wherein the first optic fiber comprises a coupler.

25. A sensor according to claim 17 wherein the second optic fiber comprises a coupler.

26. A sensor configured and arranged to releasably and operably contact a body fluid metric assessment system comprising a tunable laser and a processor, said sensor comprising:
an optic fiber having a first end that is configured and arranged to optically communicate with the tunable laser and having a second end that is configured and arranged to optically communicate with a tissue sample;
a photodiode configured and arranged to optically communicate with a tissue sample; and
a wire having a first end in electrical communication with the photodiode and a second end configured and arranged to electrically communicate with the processor.

27. A method of assessing a body fluid metric, said method comprising:
illuminating at least a portion of a body tissue with a laser;
detecting at least one wavelength of light emanating from the at least a portion of a body tissue using a single detector; and
processing the detected at least one wavelength of light emanating from the at least a portion of a body tissue to produce a body fluid metric.

28. A method according to claim 27 wherein the laser is a tunable laser.

29. A method according to claim 28 wherein the illuminating further comprises emitting light of a selected discrete wavelength from the tunable laser.

30. A method according to claim 27 wherein the illuminating further comprises emitting light from the laser toward an optical switch and selecting a wavelength of light with which to illuminate the at least a portion of a body tissue using the optical switch.

31. A method according to claim 27 wherein the illuminating further comprises emitting light from the laser toward a collimator and selecting a wavelength of light with which to illuminate the at least a portion of a body tissue using the collimator.

32. A method according to claim 27 wherein the illuminating further comprises emitting light from the laser toward a wavelength division multiplexer, splitting the emitted light into two or more beams using the wavelength division multiplexer, and illuminating a number of at least a portion of body tissues corresponding to the number of beams.

33. A method according to claim 32 wherein each at least a portion of body tissues is comprised in a single subject.

34. A method according to claim 32 wherein each at least a portion of body tissues is comprised in a separate subject.

35. A method according to claim 32 wherein the wavelength of light in each beam differs from the wavelength in the other beams.

36. A method according to claim 32 wherein the wavelength of light in each beam is the same as the wavelength in the other beams.

37. A method according to claim 27 wherein the illuminating further comprises emitting light from the laser toward an optical switch, splitting the emitted light into two or more beams using the optical switch, and illuminating a number of at least a portion of body tissues corresponding to the number of beams.

38. A method according to claim 37 wherein the wavelength of light in each beam differs from the wavelength in the other beams.

39. A method according to claim 37 wherein the wavelength of light in each beam is the same as the wavelength in the other beams.

40. A method according to claim 27 wherein the processing comprises comparing the detected at least one wavelength with a reference to form a comparison and using the comparison to determine the hydration status of the at least a portion of a body tissue.

41. A method according to claim 40 wherein the reference comprises at least a portion of an absorption spectrum for a reference tissue.

42. A method according to claim 41 wherein the comparing to form a comparison further comprises evaluating the difference between the detected at least one wavelength and at least one wavelength having a corresponding degree of absorption in the reference.

43. A method according to claim 42 wherein the evaluating further comprises quantitatively evaluating the difference between the detected at least one wavelength and the reference.

44. A method according to claim 42 wherein the evaluating further comprises qualitatively evaluating the difference between the detected at least one wavelength and the reference.

45. A method according to claim 42 wherein the absorption spectra for a reference tissue is from about 1450 nm to about 1650 nm and the reference tissue is normally hydrated tissue.

46. A method according to claim 45 wherein the comparing to form a comparison further comprises evaluating the difference between the detected at least one wavelength and the at least one wavelength having a corresponding degree of absorbance in the reference.

47. A method according to claim 46 wherein an increase in the at least one wavelength by at least a predetermined amount indicates over-hydration.

48. A method according to claim 46 wherein a decrease in the at least one wavelength by at least a pre-determined amount indicates dehydration.

49. A method according to claim 43 wherein the processing comprises comparing the detected at least one wavelength with a reference to form a comparison and using the comparison to determine the hydration status of the at least a portion of a body tissue.

50. A method according to claim 49 wherein the reference comprises at least a portion of an absorption spectrum for a reference tissue.

51. A method according to claim 50 wherein the comparing to form a comparison comprises evaluating the difference between the detected at least one wavelength and at least one wavelength having a corresponding degree of absorption in the reference.

52. A method according to claim 51 wherein the reference tissue is normally hydrated tissue.

53. A method according to claim 52 wherein the comparing to form a comparison comprises evaluating the difference between the detected at least one wavelength and the at least one wavelength having a corresponding degree of absorbance in the reference.

54. A method according to claim 53, wherein the two or more beams comprise a first beam comprising light with a wavelength of about 1390 nm and a second beam comprising light with a wavelength of about 1860 nm.

55. A method according to claim 53, wherein the two or more beams comprise a first beam comprising light with a wavelength of about 1392 nm and a second beam comprising light with a wavelength of about 1860 nm.

56. A method according to claim 53, wherein the two or more beams comprise a first beam comprising light with a wavelength of about 1380 nm, a second beam comprising light with a wavelength of about 1680 nm, and a third beam comprising light with a wavelength of about 1835 nm.

57. A method according to claim 53, wherein the two or more beams comprise a first beam comprising light with a wavelength of about 1383 nm, a second beam comprising light with a wavelength of about 1682 nm, and a third beam comprising light with a wavelength of about 1838 nm.

58. A method according to claim 53, wherein the two or more beams comprise a first beam comprising light with a wavelength of about 1395 nm, a second beam comprising light with a wavelength of about 1640 nm, a third beam comprising light with a wavelength of about 1665 nm, and a forth beam comprising light with a wavelength of about 1835 nm.

59. A method according to claim 53, wherein the two or more beams comprise a first beam comprising light with a wavelength of about 1397 nm, a second beam comprising light with a wavelength of about 1642 nm, a third beam comprising light with a wavelength of about 1667 nm, and a forth beam comprising light with a wavelength of about 1687 nm.
